(19) United States
(12) Patent Application Publication
(10) Pub. No.: US 2015/0238539 A1
(43) Pub. Date: Aug. 27, 2015

(54) COMPOSITION FOR BRAIN-NEURON PROTECTION AND BRAIN-DISEASE PREVENTION, ALLEVIATION OR TREATMENT COMPRISING MUSKRAT MUSK

(71) Applicant: Bouryeol RYU, (US)

(72) Inventor: Bouryeol Ryu, Gyeryong-si (KR)

(21) Appl. No.: 14/426,759

(22) PCT Filed: Sep. 6, 2013
(86) PCT No.: PCT/KR2013/008045

§ 371 Date: Mar. 9, 2015

(30) Foreign Application Priority Data
Sep. 7, 2012 (KR) 10-2012-0099541

(51) Int. Cl. A61K 35/48; A61K 36/232; A61K 36/40; A23L 2/52; A61K 35/644; A61K 9/28; A61K 9/48; A23L 1/30; A61K 35/28; A61K 36/258

(52) U.S. Cl. CPC A61K 35/48; A61K 35/28; A61K 36/232; A61K 36/40; A61K 36/258; A61K 35/644; A61K 9/2813; A61K 9/48; A23L 1/30; A23L 2/52; A23V 2002/00

(57) ABSTRACT

The present invention relates to a composition for the protection of brain nerve cells and the prevention, alleviation and treatment of brain diseases comprising musk from a muskrat as an active ingredient. The above composition has a superior pharmacological effect in improving nerve regeneration, suppressing damage to nerve cell, and preventing and treating Alzheimer's dementia.

### TECHNICAL FIELD

The present invention relates to a composition for the protection of brain nerve cells and the prevention, alleviation and treatment of brain diseases comprising musk from a muskrat as an active ingredient.

### BACKGROUND ART

Degenerative brain disorder is a disease where a failure of the cognitive function. occurs and induces a loss of memory powder. It also includes a variety of diseases such as dementia, Parkinson's disease, and stroke. As the population of elderly people in Korea as well as worldwide increases, various degenerative diseases of the elderly can lead to various social and economic problems. Also, in the modern society, as degenerative brain diseases such as senile dementia resulting from the aging population increases, serious social problems will come to the fore. However, the exact pathogenesis, effective prophylaxis and therapy of these diseases have not yet been developed. Dementia, one of typical degenerative brain disorders, is a disease representing the failure of the overall cognitive function. The dementia is generally caused by a chronic or progressive brain disease and appears a plurality of senior cerebral function disorders such as memory, thinking, comprehension, calculation, learning and language judgment.

Although the cause of dementia has not exactly been identified, an estimated cause includes a damage of the cholinergic nerve cells of the cerebral base, a reduction of neurotransmitters, and a beta-memory degradation caused by inflammatory responses. All these have a close relationship with the neurotransmitters synthesized from nerve cells (see Zarow, C et. al., Arch. Neural, 60, pp 337-341, 2003). The catecholamines synthesized from tyrosine in the sympathetic nerves and the indolamines synthesized from tryptophan are found in various parts of the body with their metabolites. The concentration of these substances in the nerve cells can be used as a biochemical indicator of the sympathetic nervous system function. In particular, tracing the concentration of amines and metabolites in the brain with the progress of aging and the functional ability of the receptor can help diagnose an aging brain and the related diseases. The content measurement of glutamate or GABA (gamma aminobutyric acid) in the brain is applied to the diagnosis of degenerative brain diseases or Alzheimer's diseases. This is because EAA (excitatory amino acids) such as glutamate and aspartate acts as a major excitatory neurotransmitter in the central nervous system, thereby taking an important role in various physiological actions such as a neuronal survival, synaptogenesis, learning and memory, and neuronal plasticity (see Bowen, D. M et al., Brain, 99, pp 459˜496, 1976). However, it is reported that, when accumulated in high concentrations in the extracellular fluid, they represent a neurotoxin; particularly glutamate induces hypoglycemia, status epilepticus, ischemia, hypoxia, head trauma, neuronal cell necrosis in hepatic encephalopathy, etc. (see Masotto, C et al., Phamacol. Res. Commun, 17, pp 749-772, 1985). In addition, it is reported that glutamate or GABA in the brain which is associated with degenerative brain disease and Alzheimer's disease may cause a damage to the nervous system (see Choi, D. W, J. Neurosci, 7, p 369, 1987).

Meanwhile, the senile dementia with the gradual loss of cognitive ability is associated with the activity of the cholinergic nerve cells in the central nervous system. This is known to be primarily caused by a significant reduction of the acetylcholine and choline acetyltransferase activities in the brain. Currently about 60 or more neurotransmitters have been found. It has been reported that acetylcholine, catecholamine, glutamate and GABA take an important role for various aspects of learning and memory (see Cummings J L et al., Neurology, 44, pp 2308-14, 1994).

Dementia or cognitive impairment is a disease where a normal intellectual level is maintained in the growing period, and then an acquired cognitive impairment and personality change occurs.

Cerebral nerves are destructed by a variety of causes. As such, the overall disorders of mental functions such as loss memory power, language disorders, incontinence, paranoid thinking and aphasia appear. In the course of the progress, psychiatric symptoms such as depression, personality disorder and aggression are accompanied. In the medical community, dementia caused by aging which mainly occurs in the elderly, alcoholic dementia caused by an excessive alcohol intake, and dementia caused by a genetic recessive factor expression in case were dementia rarely comes to adolescence, has been noted. However, the exact pathogenesis and therapy have not yet been identified.

Alzheimer's disease (AD) is a kind of degenerative brain diseases where a mental function is gradually weakened while the brain tissue loses its function depending on the progress of aging. Serious disorders in view of the memory power and the emotional aspect are a characteristic of this disease. In modern medicine, it is recognized to be incurable without definite therapy. Alzheimer's diseases and dementia may be regarded to be same too. However, the dementia is not only caused by the Alzheimer's diseases, but also by other adult diseases such as a hypertension, diabetes mellitus and heart disease.

This is one of the leading causes of dementia mainly appearing in the elderly. Histopathologically, this exhibits characteristics such as an overall contraction of the brain, an expansion of the ventricles, multiple lesions of nerve fibers and neuritic plaque.

### PRIOR ART DOCUMENTS

### Patent Document

(Patent Document 1) Korean Patent No. 1,160,217

(Patent Document 2) Korean Laid-open Patent Publication No. 2010-0035961

### Non-Patent Documents

(Non-Patent Document 1) Davies et al, Lancet, 21, p 1403, 1976; Rocher et al., J. Biol. Chem, 273, p 29719, 1988; Coyle et al., Science, 262, p 689, 1993

(Non-Patent Document 2) Zarow, C et al., Arch. Neural, 60, pp 337-341, 2003

(Non-Patent Document 3) Bowen, D. M. et. al., Brain, 99, pp 459˜496, 1976

(Non-Patent Document 4) Choi, D. W, J. Neurosci, 7, p 369, 1987

(Non-Patent Document 5) Cummings J L et al., Neurology, 44, pp 2308-14, 1994.

### DISCLOSURE OF INVENTION

### Technical Problem

The present inventor has identified that mice taken with a composition comprising a musk from muskrat as an active ingredient exhibit an excellent space cognitive ability. In this regard, the present inventor has found that the composition of the present invention exhibits excellent effects of the protection of brain nerve cells and the prevention, alleviation and treatment of brain diseases, thereby completing the present invention.

It is therefore an object of the present invention to provide a composition for the protection of brain nerve cells and the prevention, alleviation and treatment of brain diseases comprising musk from a muskrat which is effective for improving the nerve regeneration, suppressing the damage to the nerve cells, and preventing and treating Alzheimer's dementia;

### Technical Solution

In order to achieve the above object, the present invention provides a composition for the protection of brain nerve cells and the prevention, alleviation and treatment of brain diseases comprising musk from a muskrat as an active ingredient.

The musk refers to a musk obtained from a muskrat. The muskrat belongs to Cricetidae and its scientific name is Ondatra zibethicus. This is similar to the lemming or harvest mouse and has a body length of 15-40 cm and a tail length of about 25 cm. The muskrat is covered with a soft grayish brown hair. The snout is long and pointed, and the eyes are small. It also has long hair on tail. The body is suitable for living in water. The tail covered with scales has a long oval shape of cross sections above and below. As such, the tail is suitable for swimming and acts as a key to catching way. Its life habitat is marshes and lakes with lush aquatic plants. It is mainly active from spring till late fall. During the winter, it is less active, but does not go into hibernation. The muskrat is a herbivore, and eats tree barks, aquatic plants, reed roots, cabbages and carrots, and is bred in the form of monogamy. However, approaches to separate and breed the male in order to collect musk from the muskrat and also to breed it in the form of polygamy in the wild for reproduction have been recently studied

The musk is secreted by estrus symptoms for reproduction through the accessory reproductive gland in sachets located at the lower abdomen of the male muskrat.

The musk from a muskrat is a liquid of skin color and smells sweet. The musk is collected from the male muskrat two years after birth. Specifically, from the muskrat usually bred, the first collection is made when the bursa is hardened from March. Subsequently, the collection is made 8-10 times per year by the end of September at 15 day intervals. About 3˜5 g/year musk may usually be collected from one muskrat. The person who collects the musk from muskrat must be necessarily a person skilled for a period of time.

It is known that the general ingredients of the musk from muskrat consists of 8.46% of water, 87.0% of crude fat, 0.01% of ash, 0.024% of total saccharide, and 1% of protein, and it also contains a plurality of fragment ingredients such as normuscone, muscone, dihydrocivetone, civetone, civetol, dihydrocivetol, and dimethyl octenylcyclohexenone.

ection of brain nerve cells and the prevention, alleviation and treatment of brain diseases comprising musk from a muskrat as an active ingredient has an excellent pharmacological effect of improving the nerve regeneration, suppressing damage to nerve cells, and preventing and treating Alzheimer's dementia.

The brain diseases include Alzheimer's dementia, cerebrovascular dementia, Pick's diseases, Creutzfeldt-Jakob diseases, dementia caused by head injury or Parkinson's diseases, and preferably, Alzheimer's dementia and cerebrovascular dementia. The above brain diseases may be preferably dementia resulted from Alzheimer's diseases.

The composition of the present invention may further comprise suitable carriers, excipients and diluents which are conventionally used in the manufacture of the pharmaceutical composition.

The composition of the present invention may be formulated and used in the form of oral preparations such as powders, granules, tablets, capsules, suspensions, emulsions, syrups and aerosols as well as external preparations, suppositories and sterile injectable solutions, respectively, in accordance with conventional method. The carriers, excipients and diluents which may be used in the composition may include, for example, lactoses, dextroses, sucroses, sorbitols, mannitols, xylitols, erythritols, maltitols, starchs, acacias rubbers, alginates, gelatins, calcium phosphates, calcium silicates, celluloses, methyl celluloses, microcrystalline celluloses, polyvinylpyrrolidones, waters, methyl hydroxybenzoates, propyl hydroxybenzoates, talcs, magnesium stearates and mineral oils. When formulated, diluents or excipients, including fillers, extenders, binders, wetting agents, disintegrating agents, and surfactants, are usually used.

The solid preparation for oral administration includes tablets, pills, powders, granules, capsules and the like. These solid preparations are formulated by mixing at least one or more excipients, for example, starch, calcium carbonate, sucrose or lactose, gelatin and the like. In addition to simple excipients, lubricants such as magnesium stearate or talc are used. The liquid preparation for oral administration may include suspensions, solutions, emulsions, syrups, etc. In addition to water and liquid paraffin which are commonly used as simple diluents, various excipients, for example, wetting agents, sweeteners, fragrances, preservatives and the like may be included. Formulations for parenteral administration include sterilized aqueous solutions, nonaqueous solvents, suspensions, emulsions, lyophilized preparations and suppositories. Aqueous solvents and suspensions may include propylene glycol, polyethylene glycol, vegetable oils such as olive oils, and injectable esters such as ethyl oleates may be used. As the base of suppositories, witepsol, macrogol, tween 61, cacao butter, laurinum, glycero-gelantin and the like may be used.

The preferred dose of the compositions of the present invention varies depending on the patient's condition, body weight, severity of disease, drug form, and the administration route and period, but it may be suitably selected by those skilled in the art. However, for the preferable effect, the composition of the present invention may be administered as 1 to 1000 mg/kg per day and preferably, 50 to 500 mg/kg per day, based on healthy adults. The administration may be made once a day, and separately several times a day. The dosage is not intended to limit the scope of the invention in any aspect.

The composition of the present invention may be administered, by various routes, to mammals such as rats, mice, cattle and human. Every mode of administration may be expected. For example, the composition may be applied through oral, rectal, intravenous, intramuscular, subcutaneous, intrauterine duramater or intracerebroventricular administrations.

The musk from muskrat included in the compositions of the present invention may have a very high viscosity when it is freshly collected. In order to improve the conservative property of the musk, the musk may be diluted in a concentration of 0.00011 to 30% by weight with organic or inorganic solvents and kept under refrigeration. Preferably, the solvent may be isopropyl myristate. Upon keeping under refrigeration, the viscosity is lowered and so the suspension occurs. As such, the musk is allowed to stand at room temperature for about 5˜6 hours and then the diluted musk may be used after removing the suspension.

The musk from muskrat may be diluted in a concentration of 0.0001 to 30%. The diluted solution may be kept under refrigeration. The diluted solution kept under refrigeration stands at room temperature and it may be used after removing the suspension.

The composition of the present invention may be obtained through extraction. The extraction can be carried out by conventional extraction methods in the relevant art, e.g., room temperature extraction, heat extraction, reflux cooling extraction or ultrasonic extraction. It may be extracted using a weak acid, a weak base, a water, a C1˜C4 alcohol, or a mixture thereof as the solvent. More preferably, it may be extracted using a weak acid or an ethanol as a solvent. More preferably, the ultrasonic extraction using a weak acid as the solvent can be employed.

The musk from muskrat may be prepared by further comprising the steps of sequentially conducting a weak acid-ultrasonic extraction, a low temperature treatment and a centrifugation. The weak acid may include, but not limited to, organic acid and inorganic acid such as citric acid, lactic acid, malic acid, acetic acid, fumaric acid, and gluconic acid. Preferably, the weak acid may be KH2PO4.

The musk from muskrat may be prepared by further comprising the steps of sequentially conducting an ethanol-ultrasonic extraction, a centrifugation, and a concentration in vacuum. The composition thus extracted may be kept at room temperature and then further undergo treatments such as concentration or freeze drying.

The composition of the present invention may be a mixture of 10 parts by weight of musk from muskrat, 65 parts by weight of antler, 65 parts by weight of Angelica, 65 parts by weight of Corni fructus, 65 parts by weight of red ginseng and 180 parts by weight of honey. This mixture may be used as a preparation with spherical shape. Preferably, the mixture can be used in the form of Gongjindan composition which is coated with 0.001 to 0.1 parts by weight of pure gold having a particle size distribution of 1 to 400 nm in the above preparation.

The composition of the present invention may be a mixture of 10 parts by weight of musk from muskrat and 40 parts by weight of Panax notoginsengs. This mixture may be introduced in capsules and used in the form of a capsule preparation.

The composition of the present invention may be used in the form of health functional foods containing the above extracts as an effective ingredient showing an effect of improving a brain function and a cognitive function. The health functional food of the present invention includes tablets, capsules, pills or liquids. The foods which can add the composition of the present invention includes, for example, various foods, drinks, gums, teas, vitamin complexes, health functional foods and the like.

In addition, for the purpose of improving a brain function and a cognitive function, the composition of the present invention may be added to the foods or drinks. At this time, the above composition in the foods or drinks may be added in the amount of 0.01 to 15% by weight based on the total food weight. The health drink composition can be added at the ratio of 0.02 to 10 g and preferably 0.3 to 1 g based on the total weight of 100 ml.

The health drinks of the present invention can contain the above extracts as an essential ingredient in the indicated ratio. Additionally, the liquid ingredients can contain various flavoring agents or natural carbohydrates as additional ingredients like conventional drinks. Examples of the aforementioned natural carbohydrates include monosaccharides; for example, disaccharides such as glucose or fructose; polysaccharides such as maltose or sucrose; conventional sugars such as dextrin or cyclodextrin; and sugar alcohols such as xylitol, sorbitol or erythritol.

In addition, as the flavoring agents, natural flavors (for example, thaumatin, stevia extract (e.g., rebaudioside A, glycyrrhizin, etc.) and synthetic flavoring agents (e.g., saccharin, aspartame, etc.) may be used advantageously. The content of the natural carbohydrates is generally from about 1 to 20 g and preferably from about 5 to 12 g per 100 ml of the composition of the present invention.

Further, the compositions of the present invention can contain various nutrients, vitamins, minerals (electrolytes), flavoring agents such as synthetic flavoring agents and natural flavoring agents, coloring agents and fillers (e.g., cheese, chocolate, etc.), pectic acid and its salt, alginic acid and its salt, organic acid, protective colloidal thickeners, pH adjusting agents, stabilizers, preservatives, glycerins, alcohols, and carbonizing agents used in carbonated drinks. Furthermore, the composition of the present invention can also contain a fruit flesh for the purpose of preparing a natural fruit juice, a fruit juice beverage and a vegetable beverage. These ingredients can be used alone or in combination. The proportion of such additives can be properly adjusted, as needed, and generally may be selected in the range from 0 to about 20 parts by weight per 100 parts of the composition of the present invention.

The present invention provides a method for the protection of brain nerve cells and the prevention, alleviation and treatment of brain diseases which comprises administering the composition comprising musk from a muskrat. The abovementioned brain diseases include Alzheimer's dementia, cerebrovascular dementia, Pick's diseases, Creutzfeldt-Jakob diseases, dementia caused by head injury or Parkinson's diseases, and preferably, Alzheimer's dementia and cerebrovascular dementia. The brain diseases may be preferably a dementia resulting from Alzheimer's diseases.

For the abovementioned method for treatment, the musk from muskrat included the compositions of the present invention may have a very high viscosity when it is freshly collected. In order to improve the conservative property of the musk, the musk may be diluted in a concentration of 0.0001% to 30% by weight with organic or inorganic solvents and kept under refrigeration. Preferably, the solvent may be isopropyl myristate. Upon keeping under refrigeration, the viscosity is lowered and so the suspension occurs. As such, the musk is allowed to stand at room temperature for 5˜6 hours and the diluted musk may be used after removing the suspension.

For the abovementioned method for treatment, the musk may be diluted in a concentration of 0.0001 to 30%. The diluted solution may be kept under refrigeration. The diluted solution kept under refrigeration stands at room temperature and it may be used after removing the suspension.

For the abovementioned method for treatment, the composition of the present invention may be a mixture of 10 parts by weight of musk from muskrat, 65 parts by weight of antler, 65 parts by weight of Angelica, 65 parts by weight of Corni fructus, 65 parts by weight of red ginseng and 180 parts by weight of honey. This mixture may be used in the form of a preparation with a spherical shape. Preferably, the mixture can be used in the form of Gongjindan composition which is coated with 0.001 to 0.1 parts by weight of a pure gold having a particle size distribution of 1 to 400 nm in the above preparation.

For the abovementioned method for treatment, the compositions of the present invention may be a mixture of 10 parts by weight of musk from muskrat and 40 parts by weight of Panax notoginsengs. This mixture may be introduced in capsules and used in the form of a capsule preparation.

### Effects of the Invention

The composition according to the present invention is effective for improving a memory power and space a cognitive ability and shows a superior effect of the protection of brain nerve cells and the prevention, alleviation and treatment of brain diseases.

### DETAILED DESCRIPTION OF THE PREFERRED EMBODIMENTS

Now, the present invention will be described in detail in the following orders: Preparations, Examples and Formulation Examples. However, it should be understood that these Preparations, Examples and Formulation Examples are not intended to limit the scope of the present invention, but to aid understanding of the present invention.

Unless otherwise noted, the herbal medicines used in the Preparations, Examples and Formulation Examples were cultivated ones and purchased from the home center in the market. The reagents used herein were purchased from Sigma, Aldrich.

### PREPARATION 1

### Preparation of Musk Sample

60 g of the musk from muskrat which was collected from sachets located at the lower abdomen of 20 bred muskrats (Musk Land Co., Ltd.) was purchased. 10 g of the collected musk was used as a sample in a collected state without additional treatment.

### PREPARATION 2

### Preparation of a Diluted Musk Sample

Out of the 60 g of the musk from muskrat collected in PREPARATION 1, 30 g of the musk was diluted in a concentration of 1% by weight in isopropyl myristate (CAS NO. 110-27-0, FEMA. 3556) and kept under refrigeration for 6 hours. The diluted solution kept for refrigeration was allowed to stand at room temperature for 6 hours and then used after removing the suspension.

### PREPARATION 3

### Preparation of a Purified Musk Sample

Out of the 300 of the musk from muskrat diluted in PREPARATION 2, 20 g of the musk was mixed with 20 ml of 1.5M KH2PO4 (pH 4.5) and treated by an ultrasonic (JM Co., Ltd., ultrasonic cleaner 4020, 40 kHz) for 30 minutes. The low temperature treatment was then concentrated at a temperature of 4° C. for 2 hours. After the low temperature treatment, centrifugation was conducted at 20,000 rpm for 20 minutes to prepare the purified musk sample.

### PREPARATION 4

### Preparation of the Purified Musk Sample Extract

Out of the 20 g of musk from muskrat purified in PREPARATION 3, 10 g of the musk was mixed with 100 g of ethanol, and extracted with ultrasonic treatment for 30 minutes. The extract was centrifuged at 20, 000 rpm for 20 minutes to collect the supernatant. The ethanol-ultrasonic extraction and centrifugation were repeated five times. All of the resulting supernatant was collected and then concentrated in vacuum in order to remove ethanol. Finally, the purified musk sample was obtained as an extract.

### PREPARATION 5

### Preparation of Gongjindan Composition Containing Musk from Muskrat

Out of the 60 g of musk from muskrat collected in PREPARATION 1, 10 g of the musk was mixed with 65 g of antler, 65 g of Angelica, 65 g of Corni fructus, 65 g of red ginseng and 180 parts by weight of honey with stirring to prepare 450 g of Gongjindan composition 450 g. The Gongjindan composition was prepared in 100 spherical pills with a certain size to be 4.5 g per pill. Among the resulting 100 spherical pills, about 0.01 g of the pill per one Gongjindan composition was coated with a pulverized pure gold with greater than 99% of purity so as to have 100 nm of particle size distribution.

### PREPARATION 6

### Preparation of a Capsule Containing Musk from Muskrat

Out of the 60 g of musk from muskrat collected in PREPARATION 1, 10 g of the musk was mixed with 40 g of Panax notoginsengs to prepare 50 g of a capsule composition. The capsule composition was separately introduced in 100 capsules (Suheung Capsule, Korea) to make 500 mg per capsule, thereby obtaining a capsule preparation.

### PREPARATION EXAMPLE 1

### Tablets

50 g of the composition containing musk of PREPARATION 1 was mixed with 175.9 g of lactose, 180 g of potato starch and 32 g of colloidal silicic acid. To this mixture, 10% gelatin solution was added, pulverized and passed through a 14 mesh sieve. This was dried and then 160 g of potato starch, 50 g of talc and 5 g of magnesium stearate were added thereto to obtain a mixture in the form of tablets.

### PREPARATION EXAMPLE 2

### Injection Preparation

500 mg of the composition containing musk of PREPARATION 1 was dissolved in distilled water to make 100 ml. This solution was placed in a bottle, heated at a temperature of 20° C. for 30 minutes and then sterilized. The injection preparation was prepared in the content of 2 ml per one ampoule in accordance with a conventional method for preparing an injection preparation.

### PREPARATION EXAMPLE 3

### Health Food

1000 mg of the composition containing musk of PREPARATION 1, a suitable amount of vitamin mixture, 70 μg of vitamin A acetate, 1.0 mg of vitamin E, 0.13 mg of vitamin B1, 0.15 mg of vitamin B2, 0.5 mg of vitamin B6, 0.2 μg of vitamin B12, 10 mg of vitamin C, 10 μg of biotin, 1.7 mg of nicotinic acid amide, 50 μg of folic acid, 0.5 mg of calcium pantothenate, a suitable amount of mineral mixture, 1.75 mg of ferrous sulfate, 0.82 mg of zinc oxide, 25.3 mg of magnesium carbonate, 15 mg of potassium dihydrogen phosphate (KH₂PO₄), 55 mg of calcium monohydrogen phosphate (CaHPO₄), 90 mg of potassium citrate, 100 mg of calcium carbonate, and 24.8 mg of magnesium chloride were mixed according to a conventional method for producing a health food to obtain granules. Finally, the health food composition was prepared according to a conventional method.

The composition ratio of vitamins and mineral mixtures were designed so that the ingredients suitable for relatively healthy foods were mixed as the preferred embodiment but the mixing ratio thereof may be optionally changed.

### PREPARATION EXAMPLE 4

### Health Drinks

1000 mg of the composition containing musk of PREPARATION 1, 1000 of citric acid, 100 g of oligosaccharide, 500 mg of vitamin C, 10 mg of caramel, and purified water were added. The above ingredients were mixed in a total weight of 900 ml in accordance with a conventional method for preparing a health drink, then stirred and heated at a temperature of 85° C. for about one hour. The resulting solution was filtered, collected at sterilized 2 l container, sealed, sterilized and then kept for refrigeration, thereby obtaining the health drink of the health drink composition of the present invention.

The composition ratio of the mixture was designed so that the ingredients suitable for a relatively healthy food were mixed as the preferred embodiment, but the mixing ratio thereof may be optionally changed depending on the local or national preference such as a demand class, a demand state and an intended use.

### EXAMPLE 1

### Identification of the Efficacy of Musk against Mouse with Brain Injury

### EXAMPLE 1-1

### Preparation of Experimental Animals

As for the experimental animals, 18 to 20 g of 45 male ICR mouse purchased from DAEHAN Animal Experiment Co., Ltd. were used. Specifically, the animals were classified to include five mice per group, then placed in a cage, sufficiently fed with water and foodstuff, and bred in an animal room wherein the temperature (24 ±1° C.), relative humidity (55±5%) and the light-dark (06:00-18:00, light) were controlled during the experiment period. The animals were classified into nine groups of the non-treated group, the ethanol group, the tacrine group and the groups of PREPARATIONS 1 to 6.

### EXAMPLE 1-2

### Passive Avoidance Experiment

The passive avoidance experiment was performed in order to confirm the effects of the compositions of PREPARATIONS 1 to 6 on each memory and learning. This experiment uses the characteristics that animals intend to enter into the dark instinctively. If the animals intend to enter into the dark, an electrical stimulation is given to the animals to prevent from entering the dark. Thereafter, the short-term memory power against the electrical stimulation was measured.

Four days before the passive avoidance experiment, tacrine and substances PREPARATIONS 1 to 6 were orally administered to seven other groups except for the non-treated group and the ethanol group once a day. On day 4, the last day of administration, tacrine and compositions of PREPARATIONS 1 to 6 were orally administered. After 30 minutes, ethanol which undergoes damage to the memory power of mouse was orally administered to eight groups except for the non-treated group.

The passive avoidance experiment was conducted in the apparatus wherein each room having the same structure and 60 dB or less of noise and including a room with light and a room without light (width 20 cm, length 20 cm, height 20 cm) is connected to the guillotin door (Jungdo Bio & Plant Co. Ltd, Seoul, Korea). The mouse was placed in a bright room with shining light and observed for 10 seconds. Subsequently, the door was opened and the mouse was allowed to enter into the dark room. At this time, the time taken to go into a dark room was measured (Learning Test: acquisition trial). If the mouse enters into the dark room, the door is automatically closed. The electrical stimulation of 0.6 mA was given to the mouse through the stainless grid on the bottom for five seconds.

In order to confirm the effect of recovering the cognitive function of the composition the present invention, influencing on the long-term memory after the learning test, the mouse was placed on the bright side of the room after 24 hours. The time taken to go into the dark room (step-through latency time: time of arrival) was measured up to 300 seconds (retention trial). The results show that the longer the time taken to go into the dark, the better the passive avoidance learning and memory. The results are shown in Table 1.

TABLE 1

| | Time of arrival in | Time of arrival in |
|---|---|---|
| Sample | the learning test (s) | the retention trial (s) |
| Non-treated group | 25 | 254 |
| Ethanol | 12 | 21 |
| Tacrine + Ethanol | 20 | 85 |
| Preparation 1 + Ethanol | 25 | 250 |
| Preparation 2 + Ethanol | 27 | 243 |
| Preparation 3 + Ethanol | 25 | 247 |
| Preparation 4 + Ethanol | 24 | 251 |
| Preparation 5 + Ethanol | 23 | 248 |
| Preparation 6 + Ethanol | 26 | 238 |

As can be seen from Table 1, the oral administration of the compositions of PREPARATIONS 1 to 6 of the present invention significantly increase the time of arrival in the learning test and the maintenance test as compared to the administration of ethanol. It can also be seen that this is a remarkable numerical value even when compared with the tacrine group. Consequently, it can be confirmed that the compositions from Preparations 1 to 6 of the present invention show significantly improved effects in the learning and cognitive abilities. This phenomenon is considered that administrating the composition containing musk of the present invention led to a significant reduction in the level of memory impairment of the mouse as compared with tacrine. It can be further confirmed that the time of arrival in the learning test and the retention trial was reduced as compared to the non-treated group. The results show that the longer the time taken to enter the dark room where an electric shock was given to the experimental animal, the better the learning and cognitive abilities of passive avoidance. Accordingly, it can be confirmed that the experiment had been carried out properly.

### EXAMPLE 1-3

### Morris Water-Maze Experiment

The morris water-maze experiment was carried out to measure the degree affecting the recovery of the spatial perception ability and short-term or long-term memory power in experimental animals.

Prior to the morris water-maze experiment, tacrine and the substances of PREPARATIONS 1 to 6 were orally administered to seven groups except for the non-treated group and the ethanol group once a day. On the 4th day, the last day of administration, tacrine and the substances from Preparations 1 to 6 were administered. After 30 minutes, ethanol which undergoes damage to the memory power of the mouse was orally administered to eight groups except for the non-treated group.

In the morris water-maze apparatus, water (temperature: 22±2° C.) was filled by 32 cm within a tank filled with opaque water (diameter 120 cm, height 45 cm) and an escape platform (diameter 10 cm, height 30 cm) was installed in one section of the quadrant located 2 cm below the surface of the water. The powdered skim milk was added to be invisible. The experiment was conducted for five days. The possibility of going to the escape area by chance by changing the positions of acquisition three times a day at three different positions which were minimized. If animals go to the escape area and stay for more than 10 seconds, the time taken until an escape area was found was set to the escape latency. The mean value of the experiments 3 times a day was set to the mean escape latency. In the morris water-maze test, since the test animals remembered and find out the targeting position, the position of the marker was remained constant during the experiment period so that there was no change in the surrounding environment. In the case where the escape platform was not found within 120 seconds, the experiment animal was induced to the escape platform and stayed for 10 seconds, thereby having the position remembered.

On the last day of the experiment, the escape platform was removed. The probe test for measuring the time spent in platform quadrant was carried out. The results are shown in Table 2.

TABLE 2

| | Mean escape | Time spent in |
|---|---|---|
| Sample | latency (s) | quadrant (s) |
| Non-treated group | 21 | 38 |
| Ethanol | 121 | 18 |
| Tacrine + Ethanol | 56 | 28 |
| Preparation 1 + Ethanol | 35 | 34 |
| Preparation 2 + Ethanol | 32 | 35 |
| Preparation 3 + Ethanol | 31 | 35 |
| Preparation 4 + Ethanol | 29 | 32 |
| Preparation 5 + Ethanol | 32 | 31 |
| Preparation 6 + Ethanol | 33 | 33 |

As be seen from Table 2, for the oral administration of the compositions from Preparations 1 to 6 of the present invention, the mean escape latency was significantly reduced as compared to the administration of ethanol and that the time spent in the quadrant where present in the escape platform even after removing the escape platform was significant increased. It can be seen that this is a remarkable numerical value even when compared with the tacrine group. Consequently, it can be confirmed that the compositions from Preparations 1 to 6 of the present invention show significantly improved effects in the learning and cognitive capability as compared with tacrine. This phenomenon is considered because administrating the composition containing the musk of the present invention led to a significant reduction in the level of memory impairment of the mouse as compared to the administration of tacrine. Further, it can be confirmed that the mean escape latency of the ethanol administration group increased and the time spent in quadrant was reduced, compared with the non-treated group. The results show that the shorter the time taken for the experimental animal to find the escape platform and the longer the time taken to remember the position of the escape platform, the short-term memory power and the space cognitive ability is better. Accordingly, it can be confirmed that the experiment had been properly carried out.

### EXAMPLE 2

### Identification of the Effect of Musk against Loss of Memory, Language Disorder, and Depression

Patients, comprising 21 men and 21 women, complaining of loss of memory, language disorder and depression, were divided in 7 groups to include a total of 6 patients, each of 3 men and 3 women. The compositions from Preparations 1 to 6 of the present invention were administered to the remaining 6 groups except for the non-treated group once a day. After one week, the degree of their symptom improvement was evaluated. The evaluated items were classified into the degree of recovery of memory power, the degree of recovery of language skills, and the degree of improvement of depression. The rating of the symptom assessment was based on the non-treated group. The five-point measurement method was used to rate one point (the same as the non-treated group) to 5 points (very better than the non-treated group). The results are shown in Table 3 below.

TABLE 3

| | Degree of | Degree of | Degree of |
|---|---|---|---|
| | recovery of | recovery of | improvement |
| Sample | memory power | language skills | of depression |
| Non-treated group | 1 | 1 | 1 |
| Preparation 1 | 3.2 | 3.3 | 3.8 |
| Preparation 2 | 3.8 | 3.4 | 4.1 |
| Preparation 3 | 4.0 | 3.3 | 3.7 |
| Preparation 4 | 3.3 | 4.2 | 3.8 |
| Preparation 5 | 3.3 | 3.6 | 4.0 |
| Preparation 6 | 3.7 | 3.6 | 3.6 |

As shown in Table 3, it can be confirmed that the compositions comprising musk from muskrat of the present invention show a significant effects in improving the recovery of memory power, the recovery of language skills and the recovery of depression as compared with the non-treated group. Consequently, it can be confirmed that the composition comprising musk from muskrat of the present invention is very effective in protecting the brain nerves and preventing, alleviating and treating brain diseases such as dementia.

Therefore, the composition according to the present invention has a superior pharmacological effect in improving nerve regeneration, suppressing damage to nerve cells, and preventing and treating Alzheimer's dementia.

1. A method of protecting brain nerve cells and alleviating and treating brain diseases, comprising:
providing a composition comprising musk from a muskrat as an active ingredient; and
administering the composition to a subject.

2. The method of claim 1, wherein the brain diseases are dementia.

3. The method of claim 1, wherein the dementia is caused by Alzheimer's diseases.

4. The method of claim 1, wherein the musk is diluted in a concentration of 0.0001 to 30% by weight.

5. The method of claim 1, wherein the composition comprises 65 parts by weight of antler, 65 parts by weight of Angelica, 65 parts by weight of Corni fructus, 65 parts by weight of red ginseng and 180 parts by weight of honey, with respect to 10 parts by weight of the musk.

6. The method of claim 5, wherein the composition is an annular preparation.

7. The method of claim 6, wherein the annular preparation is Gongjindan composition which is coated with 0.001 to 0.1 parts by weight of pure gold having a particle size distribution of 1 to 400 nm.

8. The method of claim 1, wherein the composition further comprises 40 parts by weight of Panax notoginsengs with respect to 10 parts by weight of the musk.

9. The method of claim 8, wherein a capsule preparation is prepared by the composition.

10. A method of protecting brain nerve cells and improving brain diseases, comprising:
providing a heal food comprising musk from a muskrat as an active ingredient; and
administering the heal food to a subject.

11. A method of protecting brain nerve cells and improving brain diseases, comprising:
providing drinks comprising musk from a muskrat as an active ingredient; and
administering the drinks to a subject.
